(19) United States
(12) Patent Application Publication
(10) Pub. No.: US 2026/0013840 A1
(43) Pub. Date: Jan. 15, 2026

(54) SPECIMEN COLLECTION DEVICE FOR HOME SAMPLING AND TRANSPORT

(71) Applicant: Daklapack Europe B.V., Lelystad (NL)

(72) Inventors: Jelle Bart JETHS, LELYSTAD (NL); Dave Willem KLAASSEN, LELYSTAD (NL); Sander Jan VAN RUITENBEEK, LELYSTAD (NL)

(73) Assignee: Daklapack Europe B.V., Lelystad (NL)

(21) Appl. No.: 18/993,948

(22) PCT Filed: Jul. 13, 2023
(86) PCT No.: PCT/EP2023/069510

§ 371 Date: Jan. 13, 2025

(30) Foreign Application Priority Data
Jul. 14, 2022 (NL) 2032490
Jul. 14, 2022 (NL) 2032492

(51) Int. Cl. A61B 10/00

(52) U.S. Cl. CPC A61B 10/0051; A61B 10/007

(57) ABSTRACT

A specimen collection device configured to receive a donor specimen, like saliva or urine. The specimen collection device includes a tube having a collection space to receive the donor specimen which is formed by a circumferential tube wall and a tube bottom wall and a cap to close the tube after collecting the donor specimen in the collection space. The cap includes a buffer chamber to hold a buffer, which may be a liquid or tablet. The buffer chamber is closed by a seal. A cutter is positioned inside the tube to cut the seal. The specimen collection device is improved in that the cutter is positioned on an impression in the circumferential tube wall which is formed by a local deformation of the circumferential tube wall and which forms a protrusion at an inside of the tube.

This application is a U.S. National Phase filing of International Application No. PCT/EP2023/069510, filed on Jul. 13, 2023, designating the United States of America, claiming priority to and the benefit of the Dutch application NL2032490 and NL2032492, the entire contents of which are incorporated by reference.

### FIELD OF THE INVENTION

The invention relates to a specimen collection device for receiving a donor specimen, like saliva or urine. The specimen collection device comprises a tube and cap. The tube has a collection space for receiving the donor specimen. The collection space is formed by a circumferential tube wall and a tube bottom wall. The cap includes a buffer chamber for holding a buffer. The buffer chamber is covered by a seal. A cutter for cutting the seal is positioned inside the collection space of the tube. When placing the cap onto the tube, the cutter cuts the seal to let the buffer enter the collection space.

In particular, the invention relates to a home specimen collection device to allow an unskilled user collecting a donor specimen in a home situation. A collected donor specimen will directly be treated with fluid from the buffer chamber and sealed in the tube for shipping. The home specimen collection device is highly functional and can be used without complex instructions.

### BACKGROUND OF THE INVENTION

WO13106007 discloses a sample collection apparatus for collection and conveyance of biological matter. The sample collection apparatus comprises a receiving vessel and cooperating sealing cap. Each comprises a complementary mechanical interlock system, e.g. a screw thread set, operable for forming a liquid tight seal therebetween when in a coupled state. The sealing cap comprises a reservoir of fluid solution, e.g. a preserving fluid to inhibit growth of bacteria and/or fungus or a purification agent. The fluid solution is releasable from the reservoir upon coupling the sealing cap and the receiving vessel. The reservoir of the sealing cap has a thin pierceable membrane to form a sealed enclosed cavity containing the fluid therein. A knife is integrated with the receiving vessel interior walls. The knife is operably located to pierce the membrane as the sealing cap is brought into relation with the sealing vessel.

This disclosed sample collection apparatus has several drawbacks. The disclosed sample collection apparatus is designed to be handled by an unskilled user. The unskilled users screws the cap to the receiving vessel to release the fluid solution of the reservoir. The fluid solution may be a toxic fluid. For that it is important that the user cannot get in contact with the fluid solution. A drawback of the sample collection apparatus is that the unskilled user may unscrew the cap from the receiving vessel and spoil a collected fluid which might be harmful.

U.S. Pat. No. 10,576,468B2 discloses another known sample collection device for the collection of a bodily fluid which also comprises a tube and a cap. The tube is configured to receive a donor specimen. The cap includes a closed chamber holding a reagent for acting on a collected sample. The cap mates with the tube to constitute a closed sample collection device. The tube and cap are configured so that when the donor deposits the specimen and closes the tube with the cap, the chamber holding the reagent is opened to release the reagent and allow it to mix with the donor specimen. A lock device is provided that operates upon the cap reaching a predetermined close condition on the tube. The lock device may comprise a ratchet or a resilient latch which permits relative movement in a first circumferential direction while stopping movement in opposite direction.

This disclosed sample collection device has several drawbacks. The presence of the lock device may prevent an unskilled user from unscrewing the cap from the tube. However, the disclosed structure is insufficient reliable. An unskilled user may still release the cap from the tube before a lockage of the cap but after the seal of the reagent chamber being cut. A further drawback considers a poor producibility of the sample collection device in mass production.

JP6481491B2 discloses another embodiment of a sample collection device comprising a tube and a cap. The tube has a tube head for receiving a pierce element. The pierce element is a non rotatably positioned in the tube head by a stopper and ribs. After spitting, the unskilled user screws the cap containing a sealed reagent on the tube which causes a ridge at a bottom region of the cap to engage with a groove of the pierce element, such that the cap couples to the pierce element. The coupling prevents a later-on removal of the cap from the tube.

A drawback of this disclosed sample collection device is that a safety to an unskilled user to prevent the user in getting in contact with the reagent is insufficiently warranted. In addition, a producibility of the sample collection device in mass production is poor, because it requires a relative complex mould.

Other devices are disclosed in US2019/210778, KR102223879, US2004/005246, WO2021259710 an EP3220832.

Regarding the above-mentioned prior art, it is remarked that any discussion of documents, acts, materials, devices, articles or the like included in the present specification is for the purpose of providing a context for the present invention, and is not to be taken as an admission that any such matters form part of the prior art or were before the priority date of each claim of this application common general knowledge in the field relevant to the present invention.

The general object of the present invention is to at least partially eliminate the above mentioned drawback and/or to provide a usable alternative.

More specific, according to an aspect, it is an object of the invention to provide a specimen collection device which has a simple configuration contributing to a mass production by injection moulding by reducing mould complexity.

More specific, according to an aspect, it is an object of the invention to provide a specimen collection device which provides a safe use to an unskilled user, in particular in preventing an opening of the specimen collection device after collecting a donor specimen.

### SUMMARY OF THE INVENTION

In a first aspect according to the invention, the object is achieved by a specimen collection device according to the invention. The specimen collection device is arranged for receiving a donor specimen. The donor specimen is a bodily fluid e.g. a saliva or urine. The donor specimen may be a saliva e.g. for a DNA investigation, but it should be understood that the specimen collection device according to the invention is not limited to either saliva or DNA. Any biological matter such as blood, sweat, cheek swabs et cetera may cooperate with various versions of the same concept of the specimen collection device according to the invention.

The specimen collection device comprises a tube, in particular a plastic test-tube. The tube has a collection space for receiving the donor specimen. The collection space inside the tube is formed by a circumferential tube wall and a tube bottom wall.

The specimen collection device comprises a cap. The cap includes a buffer chamber for holding a buffer. The buffer chamber is closed by a rupturable seal. Here, the wording buffer is to be interpreted in a broad sense. Depending on a situation for collecting the donor specimen, a buffer in the buffer chamber may be any kind of fluid or dry solid, e.g. a small tablet. The buffer may for example be a preservative solution for preserving at least a component of the collected specimen for transport and further analysis. The buffer may include several primary components amongst which include a sterilisation compound, a lysis reagent, and optionally components of a purification system. These components may operate to stabilise and preserve, lyse, and purify matter of a biological sample such as saliva or blood.

The specimen collection device comprises a cutter for cutting the seal of the cap. The cutter is positioned inside the tube. When the cap is placed in an axial direction onto the tube, the buffer chamber is lowered into the tube opening, and the cutter cuts the seal to let the buffer enter the collection space.

The specimen collection device is improved in that the cutter is positioned on an impression in the circumferential tube wall. The impression is directed radially inwards. The impression is directed from the outside to the inside. The impression forms a recess at an outerside of the tube and a protrusion at an inside of the tube. Instead of an added rib or notch attached to the inner side of the circumferential tube wall, the presence of the impression provided as a local deformation of a circular circumferential tube wall is beneficial to simplify a production of the tube. When manufacturing the tube, no additive material may be necessary to be adhered to the inner side of the tube wall. Advantageously, the tube may be manufactured by injection moulding by using a tube mould having a simple configuration. In addition, the impression is beneficial in a temperature management in manufacturing the tubes by injection moulding, because the circumferential wall may have a substantial constant wall thickness. Further, it is an advantage that the impression at the tube wall still allows a configuration of the tube according to standardised dimensions, such that the tube remains compatible with a standardised laboratory apparatus for handling specimen tubes.

Preferably, the impression in the circumferential tube wall is directly made when moulding the tube. The tube including the impression may be moulded in a single step in which a slide-less mould is used. The tube mould may incorporate the impression. In an alternative method of manufacturing the tube, the impression may be made after moulding the tube, or by a slide mould in which during moulding process a slide moves into the mould to form the impression.

Preferably, the impression has an oblong shape in which the impression extends in an axial direction along the circumferential wall of the tube. The elongated impression may extend along a portion of the circumferential tube wall. The impression may be made locally at the tube wall. However, preferably, the impression extends from a bottom edge of the tube. An extending impression from the bottom edge of the tube is beneficial in simplifying a necessary tube mould for injection moulding the tube. At the bottom face, the impression provides a recess which may be beneficially suitable to be engaged by a tool during handling of the specimen collection device. The tool may engage in the recess to obtain a form fit which may block a rotation of the tube with respect to the tool. Herewith, the impression may contribute to a handling of the tube in a test facility, in particular in blocking a rotation of the tube when unscrewing the cap.

In an embodiment of the specimen collection device, the impression has a pocket shape, in particular a slot-shape. At the inner side of the tube, the impression has an upper end face. Preferably, the cutter is positioned on the upper end face of the impression. The cutter may be a separate item attached to the impression. Preferably, the cutter is integrally shaped with the tube. In particular, the cutter is integrally shaped with the impression. It is noted that the term ‘upper’ as an indication is to be understood for the specimen collection device in an orientation with the bottom wall downwards and the tube opening with cap upwards.

In an embodiment of the specimen collection device, information data, like a label or a logo may be provided at an outer side of the tube at the position of the impression. Readable data may be printed at the impression by a laser printer. A sticker label may be adhered to the tube at the position of the impression.

In a second aspect according to the invention, the object is achieved by a specimen collection device including a cap.

The specimen collection device is arranged for receiving a donor specimen. The donor specimen is a bodily fluid e.g. a saliva or urine. The donor specimen may be a saliva e.g. for a DNA investigation, but it should be understood that the specimen collection device according to the invention is not limited to either saliva or DNA. Any biological matter such as blood, sweat, cheek swabs et cetera may cooperate with various versions of the same concept of the specimen collection device according to the invention.

The specimen collection device comprises a tube, in particular a plastic test-tube. The tube has a collection space for receiving the donor specimen. The collection space inside the tube is formed by a circumferential tube wall and a tube bottom wall.

The cap includes a buffer chamber for holding a buffer. Here, the wording buffer is to be interpreted in a broad sense. Depending on a situation for collecting the donor specimen, a buffer in the buffer chamber may be any kind of fluid or dry solid, e.g. a small tablet. The buffer may for example be a preservative solution for preserving at least a component of the collected specimen for transport and further analysis. The buffer may include several primary components amongst which include a sterilisation compound, a lysis reagent, and optionally components of a purification system. These components may operate to stabilise and preserve, lyse, and purify matter of a biological sample such as saliva or blood.

The cap has an upper cap portion being frictionally attached by a slide element to a lower cap portion. The upper cap portion has an outer circumferential cap surface which forms a finger grip for a user to screw the cap onto the tube. The lower cap portion is provided with the buffer chamber. The lower cap portion has an outer thread being complementary shaped to an inner thread being provided in a tube head of the tube for fastening the cap to the tube. The slide element is positioned in between the upper cap portion and a lower cap portion. The slide element provides a friction based rotational attachment of the upper cap portion to the lower cap portion. When screwing the cap onto the tube, the cap will fasten until a predetermined friction force is exceeded. When exceeding the predetermined friction force, the upper cap portion starts to rotate relative to the lower cap portion. The predetermined friction force determines how strong a connection between the cap and tube will become. The slide element provides an advantage in that later counter rotation of the cap will result in a sliding of the upper cap portion relative to the lower cap portion. A stick-slip effect causes the lower cap portion to remain in position. Herewith, the cap remains connected to the tube which is beneficial in protecting an unskilled user from getting into contact with a buffer and/or collected fluid.

A major advantage of the cap out of an upper and lower cap portion being coupled by the slide element is that a one-way rotation functionality of the cap is fully incorporated in the cap itself. Herewith, the non-release functionality is incorporated in the cap. Instead of a one-way rotation functionality structured with feature on the cap and on the tube, the whole incorporation of this functionality in only the cap advantageously allows a simple configuration of the tube. The tube can remain free from structural features which otherwise should provide the one-way rotation. The tube can beneficially be manufactured by a relative simple tube mould. Herewith, the specimen collection device is improved for mass production. A further advantage is that the tube may comply to standardised requirements for specimen tubes, such that the tube can be handled by a laboratory apparatus which is arranged to deal with standardised specimen tubes.

In an embodiment of the specimen collection device, the slide element comprises a slide member at the upper cap portion and a complimentary slide member at the lower cap portion. In particular, a slide member is positioned at a bottom region of the upper cap portion and a complimentary slide member is positioned at a top region of the lower cap portion. Preferably, the slide element comprises an interlocking ridge and groove. The ridge and groove fit to each other. Each of the ridge and groove may have a circular shape to provide a friction based rotation of the upper cap portion relative to the lower cap portion.

In an embodiment of the specimen collection device, the slide element is combined with a lock element to provide a secure one-way rotation of the lower cap portion. The lock element is provided between the upper cap portion and the lower cap portion. The slide element provides a coupling of the upper cap portion to the lower cap portion in the axial direction. The lock element couples the upper cap portion to the lower cap portion whilst providing a relative rotation between these portions in a counter rotation. Herewith, the cap can be inseparable screwed onto the tube. The lower cap portion rotates together with the upper cap portion to screw the cap onto the tube, and rotates relative to the upper cap portion when the upper cap portion is counterrotated in an unscrew direction. Herewith, the lower cap portion remains on the tube and a cap removal by an unskilled user is prevented.

Preferably, the lock element comprises a ratchet having at least one ratchet element. Typically, the ratchet element has a wedge shaped member including an inclined running surface to allow the relative rotation and a stopper surface to couple the portions.

In an embodiment of the specimen collection device, the lower cap portion has a tool member to be engaged by a tool to release the lower cap portion from the tube in a test facility. In particular, the tool member is a hexagonal socket for receiving an Allen wrench. Preferably, the tool member is positioned at a top portion of the lower cap portion. The tool member is positioned above the buffer chamber. A bottom wall of the hexagonal socket may form a top wall of the buffer chamber. The seal covering the buffer chamber may be attached to a bottom end face of the lower cap portion.

In an embodiment of the specimen collection device, the lower cap portion may have a cap wall having an outer diameter fitting to an inner diameter of the collection space of the tube. When screwing the lower cap portion onto the tube, the cap wall is sliding along an inside of the tube and the seal is getting ruptured by the cutter. The corresponding outer and inner diameter is beneficial to obtain a secure cutting by the cutter.

Embodiments according to the second aspect of the invention are described by the following clauses:
1. Specimen collection device (1) for receiving a donor specimen, such as a bodily fluid (e.g. saliva or urine), which specimen collection device comprises:
   a tube (2) having a collection space (24) for receiving the donor specimen which collection space is formed by a circumferential tube wall (230) and a tube bottom wall (231);
   a cap (3) for closing the tube after collecting the donor specimen in the collection space which cap includes a buffer chamber (320) for holding a buffer, characterised in that the tube has a tube head (21) provided with a screw thread, in particular an inner thread (210), for screwing the cap onto the tube and in that the cap (3) has an upper cap portion (31) which is attached by a slide element (33) to a lower cap portion (32), wherein the upper cap portion (31) has an outer circumferential cap surface which forms a finger grip (310) for a user to screw the cap (3) onto the tube (2), wherein the lower cap portion (32) is provided with the buffer chamber (320) and a screw thread (322) being complementary shaped to the screw thread (210) of the tube for fastening the cap to the tube.
2. Specimen collection device (1) according to clause 1, wherein the slide element (33) comprises a slide member (331) at the upper cap portion (31), in particular at a bottom region, and a complimentary slide member (332) at the lower cap portion, in particular at a top region.
3. Specimen collection device (1) according to clause 1 or 2, wherein the slide element (33) comprises an interlocking ridge and groove (331, 332).
4. Specimen collection device (1) according to any of the preceding clauses, wherein a lock element (34) is provided between the upper cap portion (31) and the lower cap portion (32) to couple the upper cap portion to the lower cap portion in a screwing direction and to provide a relative rotation in a counter rotation.
5. Specimen collection device (1) according to clause 4, wherein the lock element (34) comprises a ratchet having at least one ratchet element (341, 342).
6. Specimen collection device (1) according to any of the preceding clauses, wherein the lower cap portion (32) has a top portion provided with a tool member (325), in particular a hexagonal socket for receiving an Allen wrench, for releasing the cap from the tube in a test facility.
7. Specimen collection device (1) according to any of the preceding clauses, wherein the lower cap portion (32) has a cap wall (324) having an outer diameter corresponding with an inner diameter of the collection space (24) of the tube.
8. Specimen collection device (1) according to any of the preceding clauses, wherein the seal (321) is provided at a cap end face (329) of the lower cap portion (32).
9. Specimen collection device (1) according to any of the preceding clauses, wherein the buffer chamber (320) is covered by a seal (321) and a cutter (29) for cutting the seal (321) is positioned inside the tube (2), such that when placing the cap (3) onto the tube (2), the cutter (29) cuts the seal (321) to let the buffer enter the collection space (24), wherein the cutter (29) is positioned on an impression (25) in the circumferential tube wall.
10. Specimen collection device (1) according to clause 9, wherein the impression (25) has an oblong shape, wherein the impression (25) extends in an axial direction along the circumferential tube wall (230).
11. Specimen collection device (1) according to clause 10, wherein the impression (25) extends from a bottom edge (231) of the tube.
12. Specimen collection device (1) according to clause 10 or 11, wherein the tube (2) including the impression (25) in the circumferential tube wall (230) is made in a single moulding step.
13. Specimen collection device (1) according to any of the clauses 9-12, wherein the cutter (29) is integral with the tube, in particular integral with the impression (25) of the tube (2).
14. Specimen collection device (1) according to any of the clauses 9-12, wherein the cutter (29) is attached to an upper end face (251) of the impression.
15. Specimen collection device (1) according to any of the clauses 9-14, wherein at an outer side of the tube, information data, like a label or logo, is provided at the position of the impression.
16. Non-releasable cap (3) for a specimen collection device, wherein the cap has an upper cap portion (31) which is attached by a slide element (33) to a lower cap portion (32), wherein the upper cap portion has an outer circumferential cap surface which forms a finger grip (310) for a user to screw the cap onto a tube, wherein the lower cap portion (32) is provided with the buffer chamber (320) and a screw thread (322) being complementary shaped to the screw thread (321) of the tube for fastening the cap to the tube.
17. Tube (2), in particular a plastic test-tube, for a specimen collection device (1), wherein the tube has a collection space (24) for receiving a donor specimen which collection space is formed by a circumferential tube wall (230) and a tube bottom wall (231) and a cutter (29) for cutting a seal (321) of a sealed chamber (320) of a cap (3) for closing the tube (2), in which the cutter (29) is positioned inside the collection space (24), such that when placing a cap onto the tube, the cutter (29) cuts the seal (321) to let a fluid enter the collection space, characterised in that the cutter (29) is positioned on an impression (25) in the circumferential tube wall (230).
18. Home specimen collection device (1) for collecting a donor specimen in an at home situation, wherein the home specimen collection device comprises a specimen collection device according to any of the clauses 1-15.

Thus, as shown in FIG. 2, according to the second aspect, the invention provides a specimen collection device 1 for receiving a donor specimen, such as a bodily fluid (e.g. saliva or urine). The specimen collection device comprises a tube 2 having a collection space 24 for receiving the donor specimen which is formed by a circumferential tube wall 230 and a tube bottom wall 231. The specimen collection device 1 has a cap 3 for closing the tube after collecting the donor specimen in the collection space. The cap 3 comprises an upper cap portion 31 with a finger grip and a lower cap portion 32 which is provided with the buffer chamber 320 and a screw thread 322. The cap 3 is improved in that the upper cap portion 31 is frictionally connected by a slide element 33 to the lower cap portion 32 which provides a one-way rotation functionality.

In any aspect of the invention, the specimen collection device may be further optimised. Preferably, the specimen collection device comprises a test tube being made by moulding which has standardised dimensions. Typically, the test tube is made of a plastic material. The test tube may have a standardised volume of the collection space, e.g. 3 mL. The test-tube has a basic design which fits to market standards. A standardised geometry may be maintained despite the presence of the impression at the circumferential tube wall. A test tube complying to test tube standards is beneficial in that the test tube may be compatible with all kind of machines available in the sampling industry.

In an embodiment of the specimen collection device, the tube has a tube head provided with an inner thread for attaching the cap by a screwing motion.

Further, the invention relates to a tube, in particular a plastic test-tube, for a specimen collection device, wherein the tube has a collection space for receiving a donor specimen which collection space is formed by a circumferential tube wall and a tube bottom wall and a cutter for cutting a seal of a sealed chamber of a cap for closing the tube, in which the cutter is positioned inside the collection space, such that when placing a cap onto the tube, the cutter cuts the seal to let the buffer enter the collection space, characterised in that the cutter is positioned on an impression in the circumferential tube wall.

Further, in a second aspect, the invention relates to a non-releasable cap for a specimen collection device, wherein the cap has an upper cap portion which is attached by a slide element to a lower cap portion, wherein the upper cap portion has an outer circumferential cap surface which forms a finger grip for a user to screw the cap onto the tube, wherein the lower cap portion is provided with the buffer chamber and an outer thread being complementary shaped to the inner thread of the tube for fastening the cap to the tube.

Further embodiments of the tube, cap and home specimen collection device are described above and in the dependent claims.

Further, the invention relates to a home specimen collection device for collecting a donor specimen in a home situation comprising a specimen collection device according to the invention as described above. The home specimen collection device is highly functional and can be used without complex instructions. Failure mechanisms which might render collection of a specimen defective may be eliminated or strongly reduced. The home specimen collection device is configured to effectively capture a specimen in just a few steps and just a few number of parts which is of great benefit to successfully collect and transport a donor specimen. After collecting the specimen, the tube may be placed into a specially prepared shipping box for return shipping to a test facility. A user may use the same boxes received earlier by applying a return shipping label and depositing the package the shipping service.

### BRIEF DESCRIPTION OF THE DRAWINGS

The invention will be explained in more detail with reference to the appended drawings. The drawings show a practical embodiment according to the invention, which may not be interpreted as limiting the scope of the invention. Specific features may also be considered apart from the shown embodiment and may be taken into account in a broader context as a delimiting feature, not only for the shown embodiment but as a common feature for all embodiments falling within the scope of the appended claims, in which:

FIG. 1 shows an exploded view of an embodiment of a specimen collection device according to the invention including a tube and a cap;

FIGS. 2 and 3 show an upper cap portion and a lower cap portion forming the cap in further detail;

FIG. 4 shows an enlarged cross-sectional view of the lower cap portion including a buffer chamber which is closed by a seal;

FIG. 5 shows a cross-sectional view of the tube having a sidewall impression holding a cutter at an inner side of the tube for cutting the seal of the buffer chamber; and

FIG. 6 shows a cross-sectional enlarged view of an assembled specimen collection device in which the cutter cuts the seal of the buffer chamber.

### DETAILED DESCRIPTION

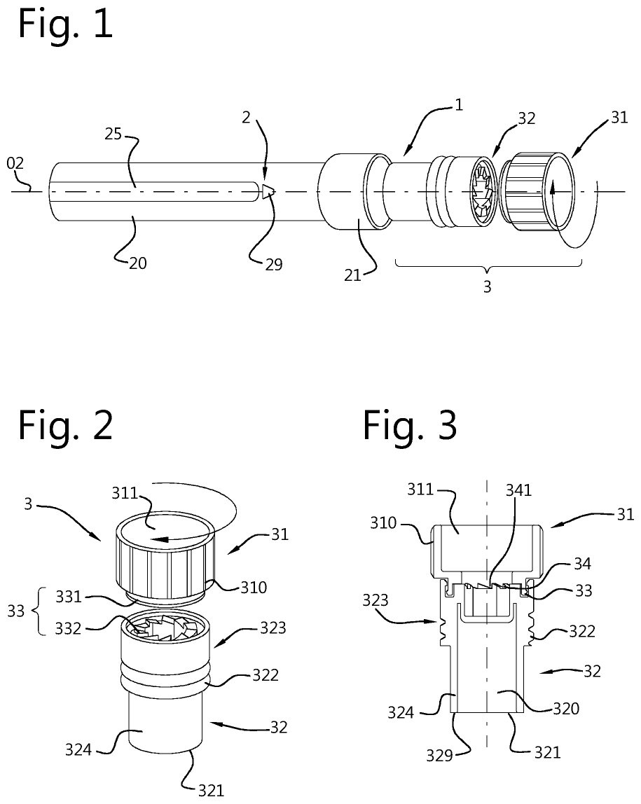

In FIGS. 1-6, an embodiment of a specimen collection device according to the invention is denoted overall by reference numeral 1. Identical reference signs are used in the drawings to indicate identical or functionally similar components. The specimen collection device is arranged for collecting a donor specimen, a bodily fluid, like saliva, sweat, blood or urine in a collection space. Here, the specimen collection device is a saliva collection device. The specimen collection device is configured for collecting a specimen in a home situation. An unskilled user may collect and ship a specimen to a test facility without a need of complex instructions.

FIG. 1 shows an exploded view of the specimen collection device 1 having a tube 2 and a cap 3. The tube 2 is a plastic test-tube. The test-tube for collecting a saliva sample typically has an inner volume of about 3 mL.

The tube 2—further shown in a cross-sectional view in FIG. 5—has a tube body 20 defining an axial axis 02 of the specimen collection device. The tube body 20 has a tube bottom 22 and a tube wall 23 which delimit a collection space 24.

The tube has a tube head 21. Here, the tube head 21 has a larger diameter than the tube body 20. The tube head 21 comprises a screw thread which is here an inner thread 210 for attaching the cap 3 to the tube by screwing.

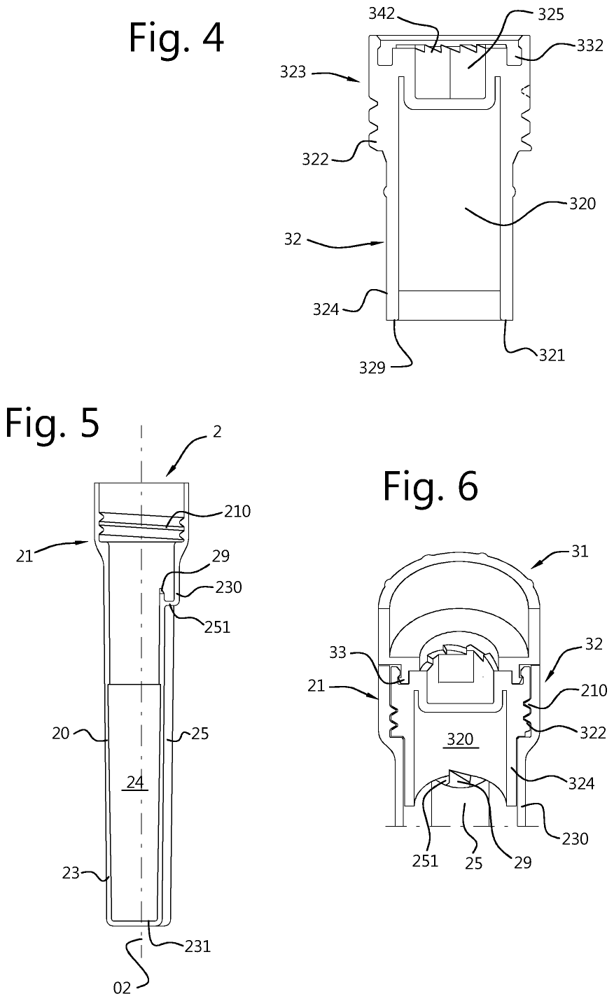

At a lower region, the cap 3 is provided with a buffer chamber which is closed by a seal, see also FIG. 4. The seal of the cap is rupturable by a cutter 29. The cutter 29 is positioned inside the tube 2. The cutter 29 has a cutter base and a cutter peak. The cutter 29 is axially directed to pierce the seal being lowered in the axial direction. When placing the cap 3 onto the tube, the cutter 29 cuts the seal of the buffer chamber to allow the buffer enter the collection space. The cutter 29 is positioned on an impression 25 in the circumferential tube wall. The impression 25 has an impression depth which is radially inwardly directed. The impression 25 presents a local deformation of the remaining circular circumferential tube wall 230. The impression 25 has an oblong shape. The elongated impression 25 has an impression length which extends in the axial direction along the circumferential tube wall. Here, the impression extends from a bottom edge 231 of the tube to the tube head.

At the outer side of the tube 2, the impression forms a recess. Information data, e.g. a label, logo or printed text may be positioned at the recess, such that the information data is visible from the outside.

The impression forms a support for supporting the cutter 29 inside the tube. As further shown in FIG. 6, here, the cutter 29 is positioned on an end face 251 of the impression 25. The cutter 29 may be a separate item attached to the impression 25 at the inside of the tube. Preferably, the cutter 29 is integral with the tube 2.

At the inside of the tube, the impression 25 spaces the cutter 29 at a distance from the circumferential tube wall 230. When placing the cap 3 on the tube, a cap wall 324 lowers in between the cutter 29 and the circumferential tube wall 230. A seal 321 positioned at a cap end face 329 will get ruptured by the cutter 29 when the cap 3 further lowers into the tube 2.

FIG. 2 further shows the cap 3 for closing the tube 2 after collecting the donor specimen in the collection space 24. The cap 3 is formed out of two portions. The cap 3 has an upper cap portion 31 and a lower cap portion 32. The upper cap portion 31 is provided with a finger grip 310 at an outer circumferential surface. The lower cap portion 32 is provided with a screw thread, here an outer thread 322, which corresponds with the inner thread 210 inside the tube head 21 of the tube 2. A user may rotate the upper cap portion 31 in a clockwise direction as indicated by the arrow in FIG. 2 to screw the cap 3 to the tube 2.

As further shown in a cross-sectional view in FIG. 3, the upper and lower cap portions, 31, 32 are interconnected by a slide element 33. The slide element 33 axially connects the upper cap portion 31 to the lower cap portion 32 whilst enabling a relative rotation in between these portions. The upper cap portion 31 is relatively rotatable with respect to the lower cap portion 32.

At a bottom side, the upper cap portion 31 is provided with a slide ridge 331 which is receivable in a slide groove 332 of the lower cap portion 32. The slide ridge 331 hooks in the slide groove 331 which axially locks the upper cap portion 31 to the lower cap portion 32. Under friction, the slide ridge 331 is movable in rotation in the slide groove 332. Due to the friction, after placing the cap 3 onto the tube 2, the lower cap portion 32 will get screwed on the tube 2. Once a screwing force exceeds the friction force, the upper cap portion 31 will rotate relative to the lower cap portion 32. Due to a stick-slip effect, a subsequent counter rotation of the upper cap portion, will leave the lower cap portion 32 in position. Herewith, the slide element 33 provides a one-way rotation cap for closing the tube. The cap 3 is made a releasable by the slide element 33.

As shown in FIGS. 2-4 and 6, the slide element 33 can be combined with a lock element 34. Here, the lock element 34 is formed by at least one ratchet. Multiple ratchets 341, 342 are annularly positioned. Each ratchet has a wedge shape including an inclined surface and a stopper surface. The inclined surface allows a relative rotation, while the stopper surface blocks the upper cap portion against the lower cap portion such that the lower cap portion rotates together with the upper cap portion.

As shown in FIG. 3, the upper cap portion 31 has a central through hole 311. The through hole 311 allows an operator at a test facility to place a release tool at the lower cap portion 32. The lower cap portion 32 has a top portion 323 which is provided with a hexagonal socket 325. The hexagonal socket 325 forms a tool member to be engaged by the release tool. The hexagonal socket 325 is arranged to receive an Allen wrench. The operator may use the Allen wrench to remove the lower cap portion 32 from the tube 2.

As further shown in the cross-sectional view of FIG. 4, the lower cap portion 32 comprises a buffer chamber 320 being closed by a seal 321. The buffer chamber 320 is arranged to store a fluid, e.g. a preservative fluid for preserving a collected donor specimen. The buffer chamber 320 is formed by a cap wall 324 which has a cap end face 329. The seal 321 is attached to the cap and face 329 to close the buffer chamber 320 at a bottom region. The buffer chamber is closed at the top region by a wall portion forming the hexagonal socket 325.

Numerous variants are possible in addition to the embodiment shown in the figures. For example, in a variant of the illustrated embodiment of the specimen collection device, the cap may be placed by snapping instead of screwing the cap onto the tube, wherein the cutter inside the tube being positioned on an impression pierces the seal of the buffer seal chamber when the cap is pressed down, such that a fluid runs out of the buffer chamber into the collection space. In a variant of the illustrated embodiment, the impression in the circumferential tube wall for supporting the cutter may be spaced apart from a bottom edge of the tube. The impression may be a local impression of the tube wall.

Although the present invention has been described in detail, it will be apparent to those skilled in the art that various changes and modifications can be made without departing from the scope of the invention as hereinafter claimed. It is intended that all such changes and modifications be encompassed within the scope of the present disclosure and claims.

Further, it is remarked that any feature of the system according to the invention which is described in the embodiments and/or mentioned in the dependent claims is in itself considered patentable without any dependency to another presented feature. In particular, any measure presented in a dependent claim is also considered patentable without dependency of the independent claim.

Thus, the invention provides in a first aspect a specimen collection device 1 for receiving a donor specimen, such as a bodily fluid (e.g. saliva or urine). The specimen collection device comprises a tube 2 having a collection space 24 for receiving the donor specimen which is formed by a circumferential tube wall 230 and a tube bottom wall 231 and a cap 3 for closing the tube after collecting the donor specimen in the collection space. The cap includes a buffer chamber 320 for holding a buffer, which may be a liquid or tablet. The buffer chamber is closed by a seal 321. A cutter 29 is positioned inside the tube 2 for cutting the seal 321. The specimen collection device 1 is improved in that the cutter 29 is positioned on an impression 25 in the circumferential tube wall.

Thus, the invention provides in a second aspect a specimen collection device 1 for receiving a donor specimen, such as a bodily fluid (e.g. saliva or urine). The specimen collection device comprises a tube 2 having a collection space 24 for receiving the donor specimen which is formed by a circumferential tube wall 230 and a tube bottom wall 231. The specimen collection device 1 has a cap 3 for closing the tube after collecting the donor specimen in the collection space. The cap 3 comprises an upper cap portion 31 with a finger grip and a lower cap portion 32 which is provided with the buffer chamber 320 and a screw thread 322. The cap 3 is improved in that the upper cap portion 31 is frictionally connected by a slide element 33 to the lower cap portion 32 which provides a one-way rotation functionality.

Reference signs list:

| | |
|---|---|
| 1 specimen collection device | 32 lower cap portion |
| 2 tube | 320 buffer chamber |
| 3 cap | 321 seal |
| 20 tube body | 322 outer thread |
| 02 axial axis | 323 top portion |
| 21 tube head | 324 cap wall |
| 210 inner thread | 325 tool member; hexagonal socket |
| 22 tube bottom | 329 cap end face |
| 23 tube wall | 33 slide element |
| 230 circumferential tube wall | 331 slide ridge |
| 231 tube bottom wall; bottom edge | 332 slide groove |
| 24 collection space | 34 lock element; ratchet |
| 25 impression | 341 first lock member; first ratchet |
| 251 impression end face | 342 second lock member; second |
| 28 information data; label; logo | ratchet |
| 29 cutter | |
| 31 upper cap portion | |
| 310 finger grip | |
| 311 through hole | |

1.-18. (canceled)

19. A specimen collection device being configured to receive a donor specimen, which specimen collection device comprises:
a tube having a collection space to receive the donor specimen which collection space is formed by a circumferential tube wall and a tube bottom wall;
a cap to close the tube after collecting the donor specimen in the collection space which cap includes a buffer chamber to hold a buffer which buffer chamber is covered by a seal; and
a cutter to cut the seal in which the cutter is positioned inside the tube to allow that when placing the cap onto the tube, the cutter to cut the seal to let the buffer enter the collection space, wherein the cutter is positioned on an impression which is formed by a local deformation of the circumferential tube wall which local deformation forms a protrusion at an inside of the tube.

20. The specimen collection device according to claim 19, wherein the impression has an oblong shape, wherein the impression extends in an axial direction along the circumferential tube wall.

21. The specimen collection device according to claim 20, wherein the impression extends from a bottom edge of the tube.

22. The specimen collection device according to claim 20, wherein the tube including the impression in the circumferential tube wall is made in a single moulding step in which a slide-less mould is used.

23. The specimen collection device according to claim 19, wherein the cutter is integral with the tube.

24. The specimen collection device according to claim 23, wherein the cutter is integral with the impression of the tube.

25. The specimen collection device according to claim 19, wherein the cutter is attached to an upper end face of the impression.

26. The specimen collection device according to claim 19, wherein at an outer side of the tube, information data is provided at the position of the impression.

27. The specimen collection device according to claim 19, wherein the cap has an upper cap portion which is attached by a slide element to a lower cap portion, wherein the upper cap portion has an outer circumferential cap surface which forms a finger grip to allow a user to screw the cap onto the tube, wherein the lower cap portion is provided with the buffer chamber and a screw thread being complementary shaped to the screw thread of the tube to allow a fastening of the cap to the tube.

28. The specimen collection device according to claim 27, wherein the slide element comprises a slide member at the upper cap portion, and a complimentary slide member at the lower cap portion.

29. The specimen collection device according to claim 27, wherein the slide element comprises an interlocking ridge and groove.

30. The specimen collection device according to claim 27, wherein a lock element is provided between the upper cap portion and the lower cap portion to couple the upper cap portion to the lower cap portion in a screwing direction and to provide a relative rotation in a counter rotation.

31. The specimen collection device according to claim 30, wherein the lock element comprises a ratchet having at least one ratchet element.

32. The specimen collection device according to claim 27, wherein the lower cap portion has a top portion provided with a tool member to release the cap from the tube in a test facility.

33. The specimen collection device according to claim 27, wherein the lower cap portion has a cap wall having an outer diameter corresponding with an inner diameter of the collection space of the tube.

34. The specimen collection device according to claim 19, wherein the seal is provided at a cap end face of the lower cap portion.

35. The specimen collection device according to claim 19, wherein the tube has a tube head provided with a screw thread to screw the cap onto the tube.

36. A plastic test-tube configured for a specimen collection device, wherein the test-tube has a collection space to receive a donor specimen which collection space is formed by a circumferential tube wall and a tube bottom wall and a cutter for cutting a seal of a sealed chamber of a cap configured to close the tube, in which the cutter is positioned inside the collection space to allow that when placing a cap onto the tube, the cutter cuts the seal to let a fluid enter the collection space, wherein the cutter is positioned on an impression which is formed by a local deformation of the circumferential tube wall and which forms a protrusion at an inside of the tube.

37. A home specimen collection device configured to collect a donor specimen in an at home situation, wherein the home specimen collection device comprises a specimen collection device being configured to receive a donor specimen, which specimen collection device comprises:
a tube having a collection space to receive the donor specimen which collection space is formed by a circumferential tube wall and a tube bottom wall;
a cap to close the tube after collecting the donor specimen in the collection space which cap includes a buffer chamber to hold a buffer which buffer chamber is covered by a seal; and
a cutter to cut the seal in which the cutter is positioned inside the tube to allow that when placing the cap onto the tube, the cutter to cut the seal to let the buffer enter the collection space, wherein the cutter is positioned on an impression which is formed by a local deformation of the circumferential tube wall which local deformation forms a protrusion at an inside of the tube.

38. The specimen collection device according to claim 21, wherein the tube including the impression in the circumferential tube wall is made in a single moulding step in which a slide-less mould is used.
